(19) United States
(12) Patent Application Publication
(10) Pub. No.: US 2018/0333250 A1
(43) Pub. Date: Nov. 22, 2018

(54) VASCULAR GRAFT FOR INTERPOSITION AT A RESECTION OF VASULAR STRUCTURES

(71) Applicant: Dokter Philippe De Vleeschauwer BV BVBA, Herselt (BE)

(72) Inventor: Phillippe DE VLEESCHAUWER, Herselt (BE)

(21) Appl. No.: 15/777,657

(22) PCT Filed: Nov. 20, 2015
(86) PCT No.: PCT/EP2015/077284

§ 371 Date: May 19, 2018

(51) Int. Cl. A61F 2/06; A61L 27/16; A61L 27/50; A61L 27/58; A61L 27/22; A61L 27/34; B33Y 80/00

(52) U.S. Cl. CPC A61F 2/06; A61L 2420/00; A61L 27/507; A61L 27/58; A61L 27/222; A61L 27/34; B33Y 80/00; A61F 2240/002; A61F 2250/0097; A61F 2210/0004; A61F 2002/065; A61F 2210/0071; A61F 2250/0039; A61F 2240/004; A61F 2230/0067; A61L 27/16

(57) ABSTRACT

The invention pertains to a vascular graft (1) for interposition at a resection of vascular structures, said vascular graft comprising a hollow body (2) elongated along a longitudinal axis (3) which includes a first end (4) defining a first opening (5) and a second end (6) defining a second opening (7), wherein said resection of vascular structures concerns a resection of the carotid bifurcation, and wherein said first end (4) is configured to be attached to the common carotid artery and said second end (6) is configured to be attached to the internal carotid artery or the external carotid artery, and wherein said vascular graft comprises polytetrafluoroethylene, and wherein said vascular graft comprises an inside diameter which decreases from said first end (4) towards said second end (6).

### TECHNICAL FIELD

The invention pertains to a use of a vascular graft for interposition at a resection of the carotid bifurcation.

### BACKGROUND

Carotid endarterectomy is the gold standard for treatment of carotid artery stenosis. Carotid endarterectomy shows the problem that it can be challenging, even technically impossible. Prosthetic carotid bypass grafting is a proven and safe alternative when carotid endarterectomy is hazardous. Nevertheless, the lack of safe and feasible alternatives for treatment of carotid stenosis can be considered as problematic.

The invention aims to resolve at least some of the problems mentioned above.

The present invention therefore aims to deliver a safe and feasible alternative for treatment of carotid stenosis.

### SUMMARY

In a first aspect, the present invention concerns a use of a vascular graft 1 for interposition at a resection of vascular structures, said vascular graft 1 comprising a hollow body 2 elongated along a longitudinal axis 3 which includes a first end 4 defining a first opening 5 and a second end 6 defining a second opening 7, wherein said resection of vascular structures concerns a resection of the carotid bifurcation, and wherein said first end 4 is configured to be attached to the common carotid artery and said second end 6 is configured to be attached to the internal carotid artery or the external carotid artery, and wherein said vascular graft 1 comprises polytetrafluoroethylene, and wherein said vascular graft 1 comprises an inside diameter which decreases from said first end 4 towards said second end 6.

The use of a vascular graft 1 for interposition at a resection of the carotid bifurcation, according to the present invention, proves to be a safe, feasible and effective means for the treatment of carotid stenosis. The use of such vascular graft 1 for interposition at a resection of the carotid bifurcation leads to clearly lower restenosis rates, shorter operating time and shorter clamping time compared to the commonly used method of carotid endarterectomy. Furthermore, polytetrafluoroethylene is an excellent material for the vascular graft 1 due to its biocompatibility and low thrombogenicity. Additionally, the differences in inside diameter between said first end 4 and said second end 6 are especially suitable for connecting with portions of the carotid arteries at level of a resection of the carotid bifurcation. Smaller inside diameters of said second end 6 are suited for portions of the internal carotid artery or external carotid artery while larger inside diameters of said first end 4 are suited for portions of the common carotid artery.

In a second aspect, the present invention concerns a vascular graft 1 comprising a hollow body 2 elongated along a longitudinal axis 3 which includes a first end 4 defining a first opening 5 and a second end 6 defining a second opening 7, which vascular graft 1 is suitable for use in a use according to the first aspect of the present invention, wherein said vascular graft 1 comprises an inside diameter which decreases from said first end 4 towards said second end 6.

### DESCRIPTION OF FIGURES

FIG. 1 shows a vascular graft 1 according to a preferred embodiment of the present invention.

FIG. 2 shows a vascular graft 1 according to a preferred embodiment of the present invention.

FIG. 3 shows a bifurcated vascular graft 1 according to a preferred embodiment of the present invention.

### DETAILED DESCRIPTION OF THE INVENTION

As used herein, the following terms have the following meanings:

The term “vascular graft” is used herein as a conduit which is suitable to be used in various vascular surgery procedures as a bridge between two blood vessels, for example two arteries or an artery and a vein. The vascular graft is preferably flexible and/or tubular. The vascular graft may be composed of synthetic materials, such as expanded polytetrafluoroethylene, polyurethane urea and/or derivatives thereof, polyethylene terephthalate and/or silicone.

The term “resection” is used herein as the excision of at least a portion of one or more vascular structures, in particular of at least a portion of the vascular structures at level of the carotid bifurcation.

The term “interposition” is used herein as the placement of an object, in particular a vascular graft 1, in between two or more body structures, in particular vascular portions at level of a resection of the carotid bifurcation.

The term “inside diameter”, as used in the present text, refers to the inside diameter of the vascular graft 1 at a cross-section which is oriented perpendicularly to said longitudinal axis 3.

The term “3D printing” refers herein to an additive manufacturing technology where a three-dimensional object is created by laying down successive layers of material. The 3D printing process generally is based on a 3D computer file or other digital representation of the volume to be filled by material. Apparatuses for performing such a 3D printing process are commonly known as 3D printers. 3D printers are generally faster, more affordable and easier to use than other additive manufacturing technologies. While the 3D printers used in professional product development are advanced and expensive, recently smaller and more affordable 3D printers have been developed that are suitable even for private use.

The term “thermoforming”, as used in the present text, refers to a process for preparing one or more shaped articles from a thermoplastic material. In thermoforming, the thermoplastic material, which can be provided as a layer of thermoplastic material, may be heated to its melting or softening point, stretched over or into a temperature-controlled single-surface or dual-surface mould and then held against or within one or more mould surfaces until the thermoformed section is sufficiently solidified such that the shaped articles formed therein retain their shape when unconstrained by the one or more mould surfaces. Thermoforming may include vacuum forming, pressure forming, etc.

The term “thermoplastic material”, as used in the present text, applies to a polymeric material that becomes pliable or moldable above a specific temperature and substantially solidifies upon cooling. Examples of thermoplastic polymeric materials or thermoplastic polymers include, but are not limited to, vinyl containing thermoplastics such as polyvinyl chloride, polyvinyl acetate, polyvinyl alcohol, and other vinyl and vinylidene resins and copolymers thereof; polyethylenes such as low density polyethylenes and high density polyethylenes and copolymers thereof; styrenes such as ABS, SAN, and polystyrenes and copolymers thereof, polypropylene and copolymers thereof; saturated and unsaturated polyesters; acrylics; polyamides such as nylon containing types; engineering plastics such as polytetrafluoroethylene, acetyl, polycarbonate, polyimide, polysulfone, and polyphenylene oxide and sulfide resins and the like.

The term “expanded polytetrafluoroethylene”, as used in the present text, refers to polytetrafluoroethylene that is expanded by an expansion process. Said expansion process produces a microporous fibrous structure which gives expanded polytetrafluoroethylene its unique properties.

In a first aspect, the present invention concerns a use of a vascular graft 1 for interposition at a resection of vascular structures, said vascular graft 1 comprising a hollow body 2 elongated along a longitudinal axis 3 which includes a first end 4 defining a first opening 5 and a second end 6 defining a second opening 7, wherein said resection of vascular structures concerns a resection of the carotid bifurcation, and wherein said first end 4 is configured to be attached to the common carotid artery and said second end 6 is configured to be attached to the internal carotid artery or the external carotid artery, and wherein said vascular graft 1 comprises polytetrafluoroethylene, and wherein said vascular graft 1 comprises an inside diameter which decreases from said first end 4 towards said second end 6.

Carotid stenosis, also called carotid artery disease, is caused by a build-up of plaque inside the wall of the carotid artery. The process of plaque buildup is called atherosclerosis. Carotid stenosis is a major risk factor for stroke and can lead to brain damage. The most common location of atherosclerotic plaque build-up is the carotid bifurcation, where the common carotid artery divides into the internal and external carotid arteries.

The use of a vascular graft 1 for interposition at a resection of the carotid bifurcation, according to the present invention, proves to be a safe, feasible and effective means for the treatment of carotid stenosis. The use of such vascular graft 1 for interposition at a resection of the carotid bifurcation leads to clearly lower restenosis rates, shorter operating time and shorter clamping time compared to the commonly used method of carotid endarterectomy. Furthermore, polytetrafluoroethylene is an excellent material for the vascular graft 1 due to its biocompatibility and low thrombogenicity. Additionally, the differences in inside diameter between said first end 4 and said second end 6 are especially suitable for connecting with portions of the carotid arteries at level of a resection of the carotid bifurcation. Smaller inside diameters of said second end 6 are suited for portions of the internal carotid artery or external carotid artery while larger inside diameters of said first end 4 are suited for portions of the common carotid artery. The use according to the present invention is not likely to be considered as obvious to a person skilled in the art, since such skilled person would use or refine the carotid endarterectomy, stenting or bypass procedures which are well-known for treatment of carotid stenosis at level of the carotid bifurcation.

In preferred embodiments, said vascular graft 1 comprises expanded polytetrafluoroethylene. Expanded polytetrafluoroethylene is known to be very waterproof and highly breathable.

In preferred embodiments, the resection of the carotid bifurcation is obtained by cutting the common carotid artery, the internal carotid artery as well as the external carotid artery at the level of the carotid bifurcation. As a result, the carotid bifurcation is removed and cut surfaces are created at level of said carotid arteries. In a preferred embodiment, the vascular graft 1 is used for interposition between the cut surface of the common carotid artery and the cut surface of the internal carotid artery, for which the first end 4 is brought into contact and attached with the cut surface of the common carotid artery while the second end 6 is brought into contact and attached with the cut surface of the internal carotid artery and while the cut surface of the external carotid artery is sealed, preferably heat sealed. In another preferred embodiment, a first vascular graft 1 is used for interposition between the cut surface of the common carotid artery and the cut surface of the internal carotid artery, for which the first end 4 is brought into contact and attached with the cut surface of the common carotid artery while the second end 6 is brought into contact and attached with the cut surface of the internal carotid artery, and a second vascular graft 1 is used for interposition between the cut surface of the external carotid artery and the first vascular graft 1, for which the second end 6 of the second vascular graft 1 is brought into contact and attached with the cut surface of the external carotid artery while the first end 4 of the second vascular graft 1 is brought into contact and attached with the first vascular graft 1. In the last mentioned embodiment, said first and second vascular grafts 1 are preferably brought into fluid communication with each other, implying that a hole should be made in the body 2 of the first vascular graft, and that the first end 4 of said second vascular graft 1 should be brought into contact and attached with said hole.

The configuration of said first 4 and second ends 6 to be attached to said carotid arteries means that said first 4 and second ends 6 are configured to be attached to the portions of said carotid arteries which remain after resection of at least a portion of the carotid arteries at level of the carotid bifurcation. Furthermore, said ends 4, 6 are configured for attachment in such a way that the interior of the hollow body 2 of the vascular graft 1 is capable of communicating with the interior of at least two of said carotid arteries, so that blood can be transported between carotid arteries through the vascular graft 1. In embodiments, said first end 4 comprises an inside diameter which is tailored or which can be tailored upon the diameter of the common carotid artery, and is thus configured to be attached to the common carotid artery. In embodiments, said second end 6 comprises an inside diameter which is tailored or which can be tailored upon the diameter of the internal carotid artery or the external carotid artery, and is thus configured to be attached to the internal carotid artery or the external carotid artery. In embodiments, the first 4 and second ends 6 are opposing each other in a normal, non-bent state of the vascular graft 1.

In a preferred embodiment, the present invention provides a use according to the first aspect of the invention, wherein one or more dimensions of said vascular graft 1 are determined by applying a medical imaging technique for the visualization of at least a portion of one or more vascular structures at level of the carotid bifurcation.

The one or more vascular structures at level of the carotid bifurcation include the common carotid artery, the internal carotid artery and the external carotid artery. In one embodiment, said visualization is applied for only one human. In preferred embodiments, said visualization is applied for multiple humans. The visualization of said vascular structures for multiple humans can be regarded as a population analysis which is representative for the dimensions of the carotid arteries at level of the carotid bifurcation. Said visualization is a most suitable and accurate means to determine one or more dimensions of said vascular graft 1.

In a preferred embodiment, the present invention provides a use according to the first aspect of the invention, wherein CT angiography is selected as a medical imaging technique.

The use of CT angiography as medical imaging technique is especially beneficial for visualization of said vascular structures at level of the carotid bifurcation, since CT angiography has a great advantage in comparison to other medical imaging techniques such as magnetic resonance tomography, positron emission tomography, single photon emission computed tomography or 3D ultrasound. Said advantage is that the entire vascular system surrounding the carotid bifurcation can be recorded in a single CT scan, by the use of a contrast agent.

In a preferred embodiment, the present invention provides a use according to the first aspect of the invention, wherein the vascular graft 1 is produced by 3D printing.

3D printing offers multiple advantages over more classical production methods, among which less waste as a result of the production process and a high production speed. 3D printing is therefore a preferred technique for the production of vascular grafts 1 according to the present invention.

In a preferred embodiment, the present invention provides a use according to the first aspect of the invention, wherein the vascular graft 1 is produced by thermoforming a thermoplastic material in a mould.

Thermoforming a thermoplastic material in a mould for the production of the vascular graft 1 has the advantage that it is a straight forward and reliable manner of producing a vascular graft 1 according to the present invention. Any suitable thermoplastic polymer as known from the state of the art, or any combinations thereof, can be selected as thermoplastic material. In a preferred embodiment, polytetrafluoroethylene is selected as thermoplastic material. In preferred embodiments, known lubricants, plasticizers, and/or processing aids are added to the thermoplastic material to lower the softening or melting point of the thermoplastic material and thus to simplify the thermoforming process.

In a preferred embodiment, the present invention provides a use according to the first aspect of the invention, wherein the vascular graft 1 is cut to desired dimensions prior to said interposition at a resection of vascular structures.

Cutting the vascular graft 1 to desired dimensions prior to said interposition at a resection of vascular structures offers a high level of flexibility to the use of the vascular graft 1 according to the present invention. In preferred embodiments, the inside diameter at the first end 4 is selected to be the same or larger than the largest common diameter of the common carotid artery in a population of humans while the inside diameter at the second end 6 is selected to be smaller than the smallest common diameter of the internal carotid artery and/or external carotid artery in a same population of humans. In this way, the simple operation of cutting the vascular graft 1 can be used to tailor the dimension of said graft 1 to the specific dimensions of said carotid arteries for any human patient.

In another embodiment, the present invention provides a use of a vascular graft 1 for interposition at a resection of vascular structures, said vascular graft 1 comprising a hollow body 2 elongated along a longitudinal axis 3 which includes a first end 4 defining a first opening 5 and a second end 6, the second end 6 being bifurcated into a first branch 13 ending in a first branch end 14 defining a first branch opening 15 and a second branch 16 ending in a second branch end 17 defining a second branch opening 18, wherein said resection of vascular structures concerns a resection of the carotid bifurcation, and wherein said first end 4 is configured to be attached to the common carotid artery, the first branch end 14 is configured to be attached to the internal carotid artery, and the second branch end 17 is configured to be attached to the external carotid artery, and wherein said vascular graft 1 comprises polytetrafluoroethylene, and wherein said vascular graft 1 comprises an inside diameter which is larger at said first end 4 than at said first 14 and second branch ends 17.

As mentioned above, the use of a vascular graft 1 for interposition at a resection of the carotid bifurcation, according to the present invention, proves to be a safe, feasible and effective means for the treatment of carotid stenosis. The use of a bifurcated vascular graft 1 with first 13 and second branches 16, according to this embodiment, furthermore provides the advantage of providing the solution of a one-piece graft 1 which can conveniently be interposed in a resection of the carotid bifurcation. All embodiments mentioned above for the use of a vascular graft 1 according to the first aspect of the present invention concerning the application of a medical imaging technique for the visualization of at least a portion of one or more vascular structures at level of the carotid bifurcation, the specific selection of CT angiography for visualization, the selection of a graft 1 produced by 3D printing or by the thermoforming of a thermoplastic material in a mould, and the cutting of the vascular graft 1 to desired dimensions prior to said interposition at a resection of vascular structures, are applicable to said vascular graft 1 comprising a second end 6 being bifurcated into first 13 and second branches 16.

In a second aspect, the present invention concerns a vascular graft 1 comprising a hollow body 2 elongated along a longitudinal axis 3 which includes a first end 4 defining a first opening 5 and a second end 6 defining a second opening 7, which vascular graft 1 is suitable for use in a use according to the first aspect of the present invention, wherein said vascular graft 1 comprises an inside diameter which decreases from said first end 4 towards said second end 6.

The differences in inside diameter between said first end 4 and said second end 6 are especially suitable for connecting with portions of the carotid arteries at level of a resection of the carotid bifurcation. Smaller inside diameters of said second end 6 are suited for portions of the internal carotid artery or external carotid artery while larger inside diameters of said first end 4 are suited for portions of the common carotid artery.

In a preferred embodiment, the present invention provides a vascular graft 1 according to the second aspect of the invention, wherein the inside diameter at said second end 6 is at most 90%, more preferably at most 80%, even more preferably at most 70%, and most preferably at most 65% of the inside diameter at said first end 4.

Such ratios between the inside diameters of the first 4 and second ends 6 provide a vascular graft 1 with a considerable variation of inside diameter from the first end 4 towards the second end 6. In this way, the first end 4 is suitable or is easily made suitable for a great variety of common carotid arteries with varying diameters while the second end 6 is suitable or is easily made suitable for a great variety of internal or external carotid arteries with varying diameters. Indeed, the diameter of said carotid arteries can vary between different individuals and can be affected by pathologies. In embodiments, said vascular graft 1 is adjusted to meet the specific diameters of said carotid arteries by cutting the body 2 of the vascular graft 1 at positions corresponding to the desired inside diameters and/or corresponding to a desired length along said longitudinal axis 3.

In a preferred embodiment, the present invention provides a vascular graft 1 according to the second aspect of the invention, wherein the inside diameter at said first end 4 is between 7 mm and 12 m and the inside diameter at said second end 6 is between 2 mm and 8 mm.

In a most preferred embodiment, the inside diameter at said first end 4 is 10 mm and the inside diameter at said second end 6 is 6 mm. In another most preferred embodiment, the inside diameter at said first end 4 is 9 mm and the inside diameter at said second end 6 is 5 mm. Said dimensions of inside diameters of the vascular graft 1 are especially suitable for employing said vascular graft 1, with or without cutting the body 2 of the vascular graft 1 at positions corresponding to desired inside diameters and/or corresponding to a desired length along said longitudinal axis 3, for interposition at a resection of the carotid bifurcation.

In a preferred embodiment, the present invention provides a vascular graft 1 according to the second aspect of the invention, wherein the vascular graft 1 comprises a tapered section 8 ending in the first end 4 and a uniformly dimensioned section 9 ending in the second end 6 and connected to said tapered section 8, in which the dimension of said tapered section 8 along said longitudinal axis 3 is at most 70%, more preferably at most 65%, even more preferably at most 60%, and most preferably at most 55% of the dimension of said uniformly dimensioned section 9 along said longitudinal axis 3.

The uniformly dimensioned section 9 can conveniently be used for dimensioning the vascular graft 1 according to said longitudinal axis 3. In embodiments, said dimensioning is performed by cutting the body 2 of the vascular graft at a position of said uniformly dimensioned section 9. Adjustment of the dimension of the vascular graft 1 along said longitudinal axis 3 confers a high extent of flexibility to said graft 1 for interposition at a resection of the carotid bifurcation, since such resection may vary in the position where said carotid arteries are cut.

In a preferred embodiment, the present invention provides a vascular graft 1 according to the second aspect of the invention, wherein the dimension of said tapered section 8 along said longitudinal axis 3 is between 3 cm and 7 cm and wherein the dimension of said uniformly dimensioned section 9 along said longitudinal axis 3 is between 8 cm and 12 cm.

In a most preferred embodiment, the dimension of said tapered section 8 along said longitudinal axis 3 is 5 cm and the dimension of said uniformly dimensioned section 9 along said longitudinal axis 3 is 10 cm. Said dimensions of the vascular graft 1 along said longitudinal axis 3 are especially suitable for employing said vascular graft 1, with or without cutting the body 2 of the vascular graft 1 at positions corresponding to desired dimensions along said longitudinal axis 3, for interposition at a resection of the carotid bifurcation.

In a preferred embodiment, the present invention provides a vascular graft 1 according to the second aspect of the invention, wherein said vascular graft 1 is a thin walled vascular graft. In other words, the body 2 of the vascular graft 1 comprises a wall with a low thickness. In preferred embodiments, said wall comprises a thickness of between 0.04 mm and 0.60 mm, more preferably of between 0.15 mm and 0.55 mm, and most preferably of between 0.18 mm and 0.50 mm.

According to these thickness levels, the vascular graft 1 shows a high flexibility for interposition at a resection of the carotid bifurcation while the graft 1 maintains sufficient strength, avoiding the accidental and undesired formation of undesired holes in the graft 1.

In a preferred embodiment, the present invention provides a vascular graft 1 according to the second aspect of the invention, wherein said vascular graft 1 comprises polytetrafluoroethylene.

In preferred embodiments, said vascular graft 1 comprises expanded polytetrafluoroethylene. Expanded polytetrafluoroethylene is known to be very waterproof and highly breathable. In preferred embodiments, the vascular graft 1 comprises polytetrafluoroethylene with a basis weight of between 0.05 g/m² and 60 g/m² and more preferably of between 0.08 g/m² and 45 g/m².

In one embodiment, the body 2 of the vascular graft 1 is formed by wrapping a layer of unsintered expanded polytetrafluoroethylene (ePTFE) tape helically such that adjacent turns of the ePTFE tape have sufficient overlap to ensure that at least 2 layers of ePTFE tape are present at any point along the length of the graft 1 along the longitudinal axis 3. In one embodiment, only two layers of tape are present.

In one embodiment, adjacent turns of said ePTFE tape overlap one another by substantially 50%, preferably 50%, of the width of the tape. In this manner, two layers of tape are present along the length of the graft 1 along the longitudinal axis, with the ends of the graft 1 having a single layer either being optionally trimmed off or allowed to remain to assist anastomosis.

In one embodiment, the vascular graft 1 includes at least two or more adjacent ePTFE tape layers wrapped in a different helical angle to each other. The tape is then sintered on a mandrel by heating above the crystalline melt point of the ePTFE. This step fuses the tape into a thin walled graft 1. After sintering the graft 1 can be removed from the mandrel. If desired, a coating layer can conveniently be applied whilst the graft 1 is still located on the mandrel.

The vascular graft 1 produced as described above may be used in a multi-layer graft, for example a tri-layer graft, or may be used alone. Optionally one of the other layers may be a fabric layer, a further self-sealing polymer or an external support member.

In one embodiment the vascular graft 1 comprises a helical external support member located thereon, for example a polyester, FEP, polytetrafluoroethylene or ePTFE beading.

An advantage of the helical tape construction is that it improves the physical properties of the graft 1, particularly the value, i.e. strength, and uniformity of suture retention.

It is possible for the vascular graft 1 to be formed from more than two layers of ePTFE tape. In one embodiment each layer of tape can be wound at a helical angle that is different to its immediately adjacent neighbouring layers. In some embodiments each layer of tape is wound at an angle which is substantially opposite to that of its immediately adjacent neighbouring layers.

In preferred embodiments, said vascular graft 1 comprises polytetrafluoroethylene which is coated with a bio-resorbable gel material, for example gelatin, on a surface thereof. Such coating of bio-resorbable gel material minimizes the formation of accidental and undesired holes in the graft 1 and provides an increase in longitudinal extensibility. The gel material for coating is preferably a bio-resorbable gelatin and any pharmaceutical grade gelatin can be used. A suitable gelatin is a mammalian gelatin. A suitable gelatin comprises a mixture of 50% normal limed bone gelatin and 50% normal gelatin treated with chloride of succinic acid. A solution of gelatin in pharmaceutical grade water is convenient for coating the grafts 1. Other gels, such as a polysaccharide gel, may also be used as a gel material for coating. Alternatively, other appropriate synthetic and biological hydrogels may be used. The preparations of such hydrogels are known in the art. Synthetic hydrogels may include poly-2-hydroxyethylmethacrylate; polyvinylalcohol; polyethylene oxide; polycarboxylic acids; poly-N-vinyl 2-pyrollidene or other synthetic hydrophilic polymers. Biological hydrogels may include starches, alginates, celluloses, agars, chitosan, collagen gels and the like.

In a preferred embodiment, the present invention provides a vascular graft 1 according to the second aspect of the invention, wherein said body 2 of the vascular graft 1 comprises one or more gradation marks 10, 11 between said first 4 and second ends 6 to indicate local inside diameters of said graft 1 along at least a portion of the graft 1, which gradation marks 10, 11 are oriented mainly perpendicular to said longitudinal axis 3.

The body 2 of the vascular graft 1 comprises an outer surface and an inner surface. The term “inner surface”, as used herein, refers to the surface of said body 2 facing towards said longitudinal axis 3. The term “outer surface”, as used herein, refers to the surface of said body 2 facing away from said longitudinal axis 3. In a preferred embodiment, said gradation marks 10, 11 are present on the outer surface of said body 2, which is advantageous for the readability of the marks. In another embodiment, said gradation marks 10, 11 are present on the inner surface of said body 2. In yet another embodiment, the gradation marks 10, 11 are present on both inner and outer surfaces. The gradation marks 10, 11 are advantageous as they visualize the inside diameters along at least a portion of the vascular graft 1. Accordingly, the vascular graft 1 can conveniently be cut to desired dimensions along or between said gradation marks 10, 11, prior to said interposition of the vascular graft 1 at a resection of the carotid bifurcation.

In a preferred embodiment, the present invention provides a vascular graft 1 according to the second aspect of the invention, wherein gradation numbers are indicated at level of one or more of said gradation marks 10, 11.

In a preferred embodiment, said gradation numbers represent the inside diameter of the vascular graft 1 at one or more positions along its body 2. In an embodiment, the gradation number “10” is used at level of the gradation mark 10 indicating an inside diameter of 10 mm, the gradation number “9” is used at level of the gradation mark 10 indicating an inside diameter of 9 mm, etc.

In a preferred embodiment, the present invention provides a vascular graft 1 according to the second aspect of the invention, wherein said body 2 of the vascular graft 1 comprises one or more longitudinal marks 12, which longitudinal marks 12 are oriented along said longitudinal axis 3.

Said longitudinal marks 12 can be used to determine if the vascular graft 1 is present in a twisted configuration. The presence of said longitudinal marks 12 along a straight line hereby indicates a non-twisted configuration of the vascular graft 1.

In a preferred embodiment, the vascular graft 1 comprises a hollow body 2 elongated along a longitudinal axis 3 which includes a first end 4 defining a first opening 5 and a second end 6, which second end 6 is bifurcated into a first branch 13 ending in a first branch end 14 defining a first branch opening 15 and a second branch 16 ending in a second branch end 17 defining a second branch opening 18, which vascular graft 1 is suitable for use in a use according to the first aspect of the present invention, wherein said first end 4, first branch end 14 and second branch end 17 comprise inside diameters, and wherein the inside diameter at said first end 4 is larger than the inside diameters at said first 14 and second branch ends 17. In a preferred embodiment, the inside diameters at said first 14 and second branch ends 17 are at most 90%, more preferably at most 80%, even more preferably at most 70%, and most preferably at most 65% of the inside diameter at said first end 4. In a preferred embodiment, the inside diameter at said first end 4 is between 7 mm and 12 mm, the inside diameter at said first branch end 14 is between 2 mm and 8 mm, and the inside diameter at said second branch end 17 is between 2 mm and 8 mm. In a most preferred embodiment, the inside diameter at said first end 4 is 10 mm and the inside diameters at said first 14 and second branch ends 17 are 6 mm. In another most preferred embodiment, the inside diameter at said first end 4 is 9 mm and the inside diameters at said first 14 and second branch ends 17 are 5 mm. Said dimensions of inside diameters of the vascular graft 1 are especially suitable for employing said vascular graft 1, with or without cutting the body 2 of the vascular graft 1 at positions corresponding to desired inside diameters and/or corresponding to a desired length along said longitudinal axis 3, for interposition at a resection of the carotid bifurcation.

The inside diameters of said first end 4 and said first 14 and second branch ends 17 are especially suitable for connecting with portions of the carotid arteries at level of a resection of the carotid bifurcation. Smaller inside diameters of said first branch end 14 are suited for portions of the internal carotid artery and smaller inside diameters of said second branch end 17 are suited for portions of the external carotid artery while larger inside diameters of said first end 4 are suited for portions of the common carotid artery. All embodiments of the second aspect of the present invention are applicable to said vascular graft 1 comprising a second end 6 which is bifurcated into first 13 and second branches 16.

In a most preferred embodiment, the present invention pertains to the use of a vascular graft 1 according to the second aspect of the present invention in a use according to the first aspect of the present invention.

In a third aspect, the present invention concerns a method to interpose a vascular graft 1 at a resection of vascular structures, the method comprising the steps of:
providing a vascular graft 1; and
placing said vascular graft 1 at a resection of vascular structures,

wherein said resection of vascular structures is a resection of the carotid bifurcation.

In a preferred embodiment, the present invention provides a method according to the third aspect of the invention, wherein the method further comprises the step of performing a resection of vascular structures, preferably the step of performing a resection of the carotid bifurcation, prior to the step of placing said vascular graft 1.

In a preferred embodiment, the present invention provides a method according to the third aspect of the invention, wherein the method further comprises the step of applying a medical imaging technique, preferably CT angiography, on one or more human patients for the visualization of at least a portion of one or more vascular structures at level of the carotid bifurcation. This step is preferably carried out prior to the step of providing the vascular graft 1. In this way, dimensions of the vascular graft 1 can be optimized according to the visualized dimensions of the vascular structures at level of the carotid bifurcation.

In a preferred embodiment, the present invention provides a method according to the third aspect of the invention, wherein the step of providing the vascular graft 1 comprises the production of the vascular graft 1. In preferred embodiments, the vascular graft 1 is produced according to dimensions of vascular structures at level of the carotid bifurcation.

In a preferred embodiment, the present invention provides a method according to the third aspect of the invention, wherein the method further comprises the step of re-sizing the vascular graft 1, prior to the step of placing said vascular graft 1. In preferred embodiments, the re-sizing is carried out by cutting a vascular graft 1 to a graft 1 of desired dimensions, such desired dimensions being dependent from the dimensions of one or more vascular structures at level of the carotid bifurcation.

In a preferred embodiment, the present invention provides a method according to the third aspect of the invention, wherein the step of providing a vascular graft 1 concerns providing a vascular graft 1 according to the second aspect of the present invention.

### EXAMPLES

### Example 1

A total of 153 consecutive carotid artery procedures were performed between January 2007 and October 2014 on 147 patients, as described in the scientific publication by Y. Mandeville, E. Canovai, I. Diebels, R. Suy and P. De Vleeschauwer, Annals of Vascular Surgery, Volume 29, Issue 8, P. 1589-1597. Data collection and statistical analysis were performed retrospectively. The outcome of patients who underwent conventional carotid endarterectomy was compared with the outcome of those who had undergone interposition of a vascular graft at a resection of the carotid bifurcation. Operating or procedure time was defined as time from induction until extubation. The first procedures of interposition of a vascular graft at a resection of the carotid bifurcation were performed in technical challenging cases such as restenosis requiring reintervention and pseudoaneurysms of the internal carotid artery. With increasing experience, interposition of a vascular graft at a resection of the carotid bifurcation was performed more routinely. Toward the end of the study, interposition of a vascular graft at a resection of the carotid bifurcation had become the procedure of choice. All patients presented for follow-up 1 month postoperatively, and then, yearly for routine physical examination and duplex ultrasound. In case of a peak systolic velocity greater than 120 cm/sec, we performed a computed tomography (CT) angiography to confirm the restenosis. If CT was contra-indicated, a magnetic resonance (MR) angiography was performed. Restenosis was defined as a 50% decrease in vessel diameter.

The effect of procedure type on rate of restenosis was expressed in terms of the odds ratio (OR) with 95% confidence intervals. An additional logistic regression model was constructed to assess probability of restenosis with inclusion of potential predictors. Potential risk factors are age, sex, procedure time, and clamping time. Clamping time and total procedure or operating time were compared using the nonparametric Wilcoxon test. For clamping time, patients who received a shunt were excluded. In case of bifurcated interposition of a vascular graft at a resection of the carotid bifurcation procedures, total clamping was used in the analysis. Restenosis free survival time was calculated with a Kaplan-Meier survival curve and compared during a 6-year follow-up period. The log-rank test was used to compare the survival times in both treatment groups. The effect of the procedure on postoperative restenosis was estimated based on a Cox proportional hazard model.

Concerning the surgical technique, a patient was placed in the standard position for carotid artery surgery that is in the supine position with the head extended and rotated to the contralateral side. The classic approach to the carotid bifurcation including wide exposure of the distal internal carotid artery was undertaken. After systemic heparinization (50 IU/kg), the internal carotid artery was clamped, followed by clamping of the common carotid artery and the external carotid artery. If the decision was made to perform an interposition of a vascular graft at a resection of the carotid bifurcation, the superior thyroid artery was clipped, and the external carotid artery ligated with 5-0 polypropylene (Prolene, Ethicon™, Amersfoort, The Netherlands). The carotid bifurcation was carefully denudated, and care was taken not to damage the carotid body, vagal nerve, or other autonomous nerve structures. The bifurcation was then completely resected, and a polytetrafluoroethylene interposition graft was prepared. We used a 6-mm polytetrafluoroethylene thin wall vascular graft (Gore-Tex Vascular™, Newark; Vascutek, Inchinnan, UK). First, an angled anastomosis was made between the internal carotid artery and the polytetrafluoroethylene graft using a Gore Tex-CV6 suture. Afterward, the proximal anastomosis was created between the graft and the common carotid artery. Both anastomoses were fashioned as end-to-end running sutures. A shunt was never used in the interposition of a vascular graft at a resection of the carotid bifurcation cases regardless of backflow, whereas a shunt was used in 11 (20%) carotid endarterectomy cases, when there was no visible, pulsatile backflow from the internal carotid artery. Stump pressures were never measured. Patch angioplasty was performed in all cases using a Dacron patch. In case of contralateral occlusion of the internal carotid artery or when the patient underwent a previous contralateral interposition of a vascular graft at a resection of the carotid bifurcation procedure, the external carotid artery was reimplanted. After the initial interposition between the internal carotid artery and the common carotid artery, a second interposition graft was placed via a proximal side-to-end anastomosis between the two grafts and a distal end-to-end anastomosis between the graft and the external carotid artery. Postoperatively, the patients were admitted to the intensive care unit for 24 hr of close neurologic and hemodynamic monitoring. All patients were given oral antiplatelet therapy (160 mg acetylsalicylic acid) and a statin.

A total of 103 interposition of a vascular graft at a resection of the carotid bifurcation procedures (67.3%) and 50 carotid endarterectomy procedures (32.7%) were performed. Overall, 10.7% (n=11) of the interposition of a vascular graft at a resection of the carotid bifurcation procedures were bifurcated interposition of a vascular graft at a resection of the carotid bifurcation procedures. During the study period, an increase in interposition of a vascular graft at a resection of the carotid bifurcation procedures and a decrease in conventional carotid endarterectomy procedures were noted. Median age at time of surgery was 70.4 years (range, 42-87 years). The degree of stenosis was classified into two groups, 50-79% and 80-99% stenosis. The vast majority of procedures were for occlusive disease (98.7%) with only two procedures because of pseudoaneurysms (1.3%), both after previous carotid endarterectomy. The 30-day mortality was 1% for the interposition of a vascular graft at a resection of the carotid bifurcation group and 0% in the carotid endarterectomy group (P value, 0.4839), with an overall 30-day mortality of 0.7%. Twelve patients died during long-term follow-up (>30 days). Disease-related (cardiovascular) and nondisease related mortality rates were 5 (3.3%) and 8 patients (5.2%), respectively (OR=0.4167, P=0.5121). Multivariate analyses result in an intercept-only model implying that there are no risk factors for postoperative mortality identified. The cerebral or local complications were comparable in both groups. The 30-day stroke rate was 1.9% for the interposition of a vascular graft at a resection of the carotid bifurcation group and 0% in the carotid endarterectomy group (P value, 0.3222).

Nerve damage (hypoglossal, facial, vagal) was not included because of insufficient data due to the retrospective nature of the study. Median follow-up time was 29.1 months with mean follow-up of 46.7 months for carotid endarterectomy and 20.7 months for interposition of a vascular graft at a resection of the carotid bifurcation. A restenosis rate of 16.0% (8/50) was observed in the carotid endarterectomy group. In the interposition of a vascular graft at a resection of the carotid bifurcation group, there was a restenosis rate of 1.9% (2/103; OR, 0.1040 [0.02118−0.5102]; P=0.0053). Both restenosis were at the proximal anastomosis, not the distal. All restenoses were asymptomatic. The estimated probability of restenosis after surgery equals 1.94% and 18% in the interposition of a vascular graft at a resection of the carotid bifurcation group and carotid endarterectomy group, respectively. The Kaplan-Meier estimate for restenosis was 6.3% in the carotid endarterectomy group and 2.1% in the interposition of a vascular graft at a resection of the carotid bifurcation group after 750 days. Based on the log-rank test, the restenosis-free survival times are not significantly larger in the interposition of a vascular graft at a resection of the carotid bifurcation group compared with the carotid endarterectomy group (chi-square=3.6 on 1 degree of freedom, P=0.0578). There is not enough evidence to prove significant differences in the restenosis-free survival times between the two groups. Mean clamping time in the carotid endarterectomy group and interposition of a vascular graft at a resection of the carotid bifurcation group was 40.10 and 34.57 min, respectively. Clamping time was compared between the interposition of a vascular graft at a resection of the carotid bifurcation group and the carotid endarterectomy procedures that did not receive a shunt. Mean operating time in the carotid endarterectomy group and interposition of a vascular graft at a resection of the carotid bifurcation group was 128.6 and 111.74 min, respectively. Both procedure or operating time and clamping time were significantly shorter in the interposition of a vascular graft at a resection of the carotid bifurcation group (P=0.01375 and P=0.000408).

Example 1 illustrates that the use of a vascular graft 1 for interposition at a resection of the carotid bifurcation, according to the present invention, proves to be a safe, feasible and effective means for the treatment of carotid stenosis. The use of such vascular graft 1 for interposition at a resection of the carotid bifurcation leads to clearly lower restenosis rates, shorter operating time and shorter clamping time compared to the commonly used method of carotid endarterectomy.

### Example 2

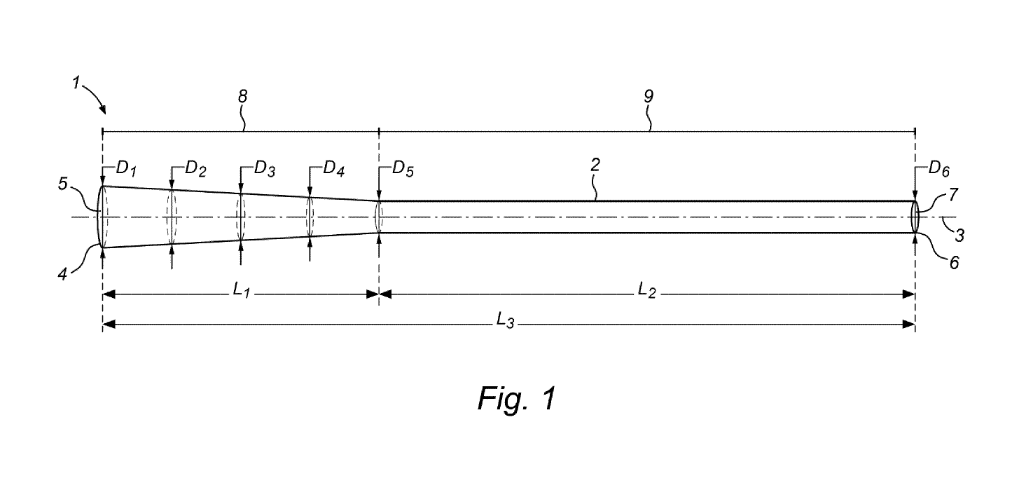

FIG. 1 shows a vascular graft 1 according to a preferred embodiment of the present invention. The vascular graft 1 comprises a hollow body 2 which is elongated along a longitudinal axis 3. The vascular graft 1 ends in a first end 4 defining a first opening 5 and a second end 6 defining a second opening 7. The vascular graft 1 comprises an inside diameter which decreases from said first end 4 towards said second end 6. Furthermore, the vascular graft comprises a tapered section 8 ending in the first end 4 and a uniformly dimensioned section 9 ending in the second end 6 and connected to said tapered section 8. The inside diameter of said tapered section 8 decreases from said first end 4 towards said uniformly dimensioned section 9. In FIG. 1, the inside diameter is indicated along five positions of the tapered section 8, which inside diameters at those positions are indicated with D1, D2, D3, D4 and D5. The uniformly dimensioned section 9 presents a uniform inside diameter which is indicated with D6 in FIG. 1. Besides, the length of the tapered section 8 along the longitudinal axis 3 is indicated with L1 in FIG. 1 while the length of the uniformly dimensioned section 9 along said longitudinal axis 3 is indicated with L2. The length of the vascular graft 1 along the longitudinal axis 3 is indicated with L3 in FIG. 1. L1 corresponds to 5 cm, L2 corresponds to 10 cm and L3 corresponds to 15 cm while D1, D2, D3, D4, D5 and D6 are corresponding to 10 mm, 9 mm, 8 mm, 7 mm, 6 mm and 6 mm, respectively. The mentioned inside diameters (D1, D2, D3, D4, D5 and D6) and lengths (L1, L2 and L3) along the longitudinal axis 3 of the vascular graft 1 are especially suitable for employing the vascular graft 1, with or without cutting the body 2 of the vascular graft 1 at positions corresponding to desired inside diameters and/or corresponding to a desired length along said longitudinal axis 3, for interposition at a resection of the carotid bifurcation.

### Example 3

In another preferred embodiment, the present invention provides a vascular graft 1 as shown in FIG. 1 wherein L1 corresponds to 5 cm, L2 corresponds to 10 cm and L3 corresponds to 15 cm while D1, D2, D3, D4, D5 and D6 are corresponding to 9 mm, 8 mm, 7 mm, 6 mm, 5 mm and 5 mm, respectively.

### Example 4

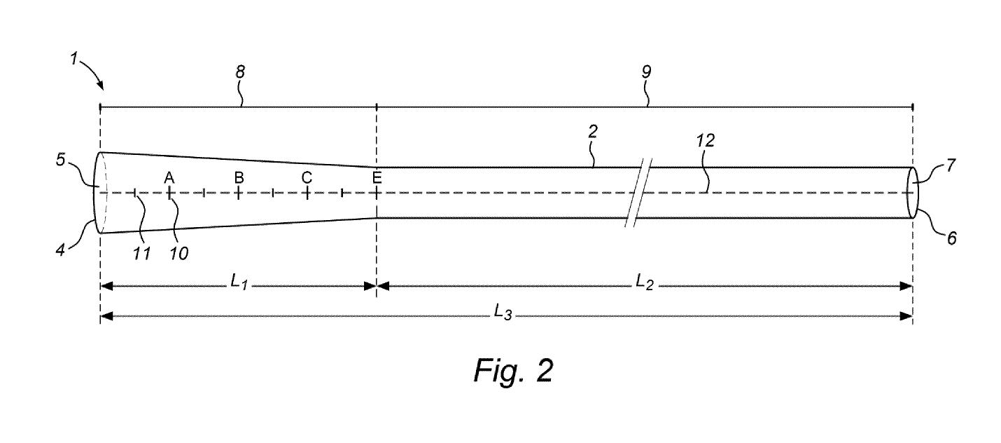

FIG. 2 shows a vascular graft 1 according to a preferred embodiment of the present invention. The outer surface of the tapered section 8 of the vascular graft 1 shows multiple gradation marks 10, 11 which are oriented mainly perpendicular to said longitudinal axis 3. The gradation marks 10, 11 are advantageous as they visualize the inside diameters along the tapered section 8 of the vascular graft 1. Accordingly, the vascular graft 1 can conveniently be cut to desired dimensions along or between said gradation marks 10, 11, prior to an interposition of the vascular graft 1 at a resection of the carotid bifurcation. A distinction is made between larger gradation marks 10 indicating inside diameters corresponding to a whole mm value and smaller gradation marks 11 indicating inside diameters corresponding to a halve mm value. Besides, the outer surface of the vascular graft 1 as presented in FIG. 2 shows longitudinal marks 12 which are oriented along said longitudinal axis 3. The longitudinal marks 12 can conveniently be used to determine if the vascular graft 1 is present in a twisted or non-twisted configuration. On the outer surface of the vascular graft 1, gradation numbers are indicated above each of the larger gradation marks 10. In FIG. 2, these gradation numbers are indicated with A, B, C and E. The gradation numbers are, next to the gradation marks 10, 11, convenient means for the visual determination of inside diameters of the vascular graft 1. In this embodiment, A corresponds to the number “9” while B, C and E are corresponding to the number “8”, the number “7” and the number “6”, respectively.

The inside diameters (D1, D2, D3, D4, D5 and D6) as well as the lengths (L1, L2 and L3) along the longitudinal axis 3 as mentioned for example 2 correspond to the inside diameters and lengths along the longitudinal axis 3 for the embodiment of example 4.

### Example 5

In this embodiment, the gradation number A of the vascular graft 1 as shown in FIG. 2 corresponds to the number “8” while B, C and E are corresponding to the number “7”, the number “6” and the number “5”, respectively.

The inside diameters (D1, D2, D3, D4, D5 and D6) as well as the lengths (L1, L2 and L3) along the longitudinal axis 3 as mentioned for example 3 correspond to the inside diameters and lengths along the longitudinal axis 3 for the embodiment of example 5.

### Example 6

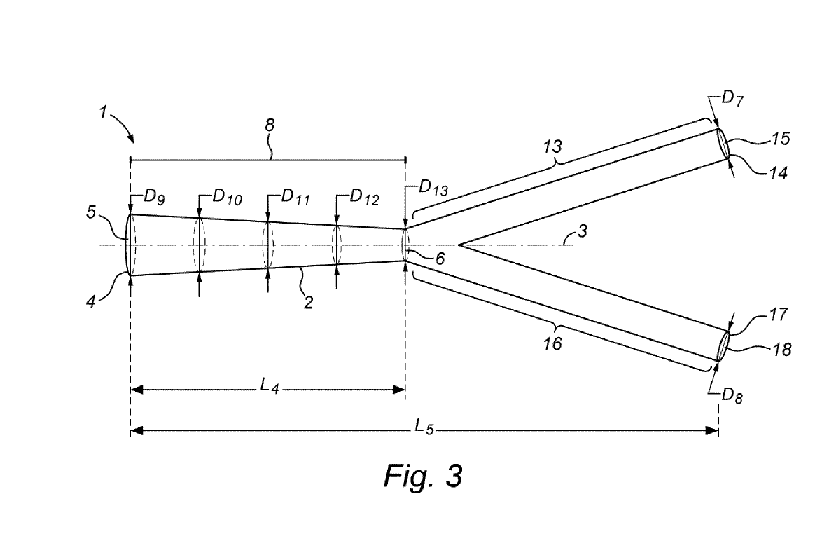

FIG. 3 shows a bifurcated vascular graft 1 according to a preferred embodiment of the present invention. The vascular graft 1 comprises a hollow body 2 elongated along a longitudinal axis 3 and includes a first end 4 defining a first opening 5 and a second end 6, which second end 6 is bifurcated into a first branch 13 ending in a first branch end 14 defining a first branch opening 15 and a second branch 16 ending in a second branch end 17 defining a second branch opening 18. The vascular graft 1 comprises a tapered section 8 which is defined between said first end 4 and said second end 6. The tapered section 8 comprises an inside diameter which decreases from said first end 4 towards said second end 6. As well the first branch 13 as the second branch 16 have a constant inside diameter. In FIG. 3, the inside diameter is indicated along five positions of the tapered section 8, which inside diameters at those positions are indicated with D9, D10, D11, D12 and D13. The uniformly dimensioned first 13 and second branches 16 present uniform inside diameters which are indicated with D7 and D8, respectively. Besides, the length of the tapered section 8 along the longitudinal axis 3 is indicated with L4 in FIG. 3 while the length of the vascular graft 1 along the longitudinal axis 3 is indicated with L5 in FIG. 3. L4 corresponds to 5 cm and L5 corresponds to 15 cm while D7, D8, D9, D10, D11, D12 and D13 are corresponding to 6 mm, 6 mm, 10 mm, 9 mm, 8 mm, 7 mm and 6 mm, respectively.

The mentioned inside diameters (D7, D8, D9, D10, D11, D12 and D13) and lengths (L4 and L5) along the longitudinal axis 3 of the vascular graft 1 are especially suitable for employing the vascular graft 1, with or without cutting the body 2 of the vascular graft 1 at positions corresponding to desired inside diameters and/or corresponding to a desired length along said longitudinal axis 3, for interposition at a resection of the carotid bifurcation.

### Example 7

In another preferred embodiment, the present invention provides a bifurcated vascular graft 1 as shown in FIG. 3 wherein L4 corresponds to 5 cm and L5 corresponds to 15 cm while D7, D8, D9, D10, D11, D12 and D13 are corresponding to 5 mm, 5 mm, 9 mm, 8 mm, 7 mm, 6 mm and 5 mm, respectively.

1. Use of a vascular graft (1) for interposition at a resection of vascular structures, said vascular graft (1) comprising a hollow body (2) elongated along a longitudinal axis (3) which includes a first end (4) defining a first opening (5) and a second end (6) defining a second opening (7), characterized in that said resection of vascular structures concerns a resection of the carotid bifurcation, and that said first end (4) is configured to be attached to the common carotid artery and said second end (6) is configured to be attached to the internal carotid artery or the external carotid artery, and that said vascular graft (1) comprises polytetrafluoroethylene, and that said vascular graft (1) comprises an inside diameter which decreases from said first end (4) towards said second end (6).

2. Use according to claim 1, characterized in that one or more dimensions of said vascular graft (1) are determined by applying a medical imaging technique for the visualization of at least a portion of one or more vascular structures at level of the carotid bifurcation.

3. Use according to claim 2, characterized in that CT angiography is selected as a medical imaging technique.

4. Use according to any of the claims 1 to 3, characterized in that the vascular graft (1) is produced by 3D printing.

5. Use according to any of the claims 1 to 3, characterized in that the vascular graft (1) is produced by thermoforming a thermoplastic material in a mould.

6. Use according to any of the claims 1 to 5, characterized in that the vascular graft (1) is cut to desired dimensions prior to said interposition at a resection of vascular structures.

7. Vascular graft (1) comprising a hollow body (2) elongated along a longitudinal axis (3) which includes a first end (4) defining a first opening (5) and a second end (6) defining a second opening (7), which vascular graft (1) is suitable for use in any of the preceding claims, characterized in that said vascular graft (1) comprises an inside diameter which decreases from said first end (4) towards said second end (6).

8. Vascular graft (1) according to claim 7, characterized in that the inside diameter at said second end (6) is at most 90% of the inside diameter at said first end (4).

9. Vascular graft (1) according to claim 7 or 8, characterized in that the inside diameter at said first end (4) is between 7 mm and 12 m and the inside diameter at said second end (6) is between 2 mm and 8 mm.

10. Vascular graft (1) according to any of the claims 7 to 9, characterized in that the vascular graft (1) comprises a tapered section (8) ending in the first end (4) and a uniformly dimensioned section (9) ending in the second end (6) and connected to said tapered section (8), in which the dimension of said tapered section (8) along said longitudinal axis (3) is at most 70% of the dimension of said uniformly dimensioned section (9) along said longitudinal axis (3).

11. Vascular graft (1) according to claim 10, characterized in that the dimension of said tapered section (8) along said longitudinal axis (3) is between 3 cm and 7 cm and that the dimension of said uniformly dimensioned section (9) along said longitudinal axis (3) is between 8 cm and 12 cm.

12. Vascular graft (1) according to any of the claims 7 to 11, characterized in that said vascular graft (1) comprises polytetrafluoroethylene.

13. Vascular graft (1) according to any of the claims 7 to 12, characterized in that said body (2) of the vascular graft (1) comprises one or more gradation marks (10, 11) between said first (4) and second ends (6) to indicate local inside diameters of said graft (1) along at least a portion of the graft (1), which gradation marks (10, 11) are oriented mainly perpendicular to said longitudinal axis (3).

14. Vascular graft (1) according to claim 13, characterized in that gradation numbers are indicated at level of one or more of said gradation marks (10, 11).

15. Vascular graft (1) according to any of the claims 7 to 14, characterized in that said body (2) of the vascular graft (1) comprises one or more longitudinal marks (12), which longitudinal marks (12) are oriented along said longitudinal axis (3).
